(19) United States
(12) Patent Application Publication
(10) Pub. No.: US 2025/0268909 A1
(43) Pub. Date: Aug. 28, 2025

(54) Combination Therapy

(71) Applicant: US Oncology Corporate, Inc., Las Colinas, TX (US)

(72) Inventor: Joyce O'Shaughnessy, Dallas, TX (US)

(21) Appl. No.: 18/858,521

(22) PCT Filed: Apr. 19, 2022
(86) PCT No.: PCT/US2022/025378

§ 371 Date: Oct. 21, 2024

(51) Int. Cl. A61K 31/55; A61K 31/337; A61P 35/00

(52) U.S. Cl. CPC A61K 31/55; A61K 31/337; A61P 35/00

(57) ABSTRACT

This disclosure relates to methods of treating breast cancer by using a combination of alisertib and paclitaxel.

### TECHNICAL FIELD

This disclosure relates to methods of treating breast cancers using a combination of alisertib and paclitaxel.

### BACKGROUND

Breast cancer is a heterogeneous disease, with distinct subtypes distinguished by expression of hormone receptors and human epidermal growth factor receptor 2 (ERBB2, formerly HER2), as well as by characteristic gene expression profiles. Triple-negative (TN) breast cancer is defined by the absence of estrogen (ER), progesterone (PgR), and HER2 receptor over expression. Compared with other breast cancer subtypes, TN breast cancer (TNBC) is associated with a worse prognosis, including a shorter time to recurrence in early-stage disease and a shorter time between recurrence and death in the metastatic setting. Metastatic ER+ and TNBCs develop progressive resistance to anti-cancer therapies leading to tumor progression and death.

Aurora Kinase A (AURKA) is a serine/threonine kinase that regulates the transition from G2 to mitosis, and is itself regulated by the pro-proliferative cell cycle transcription factor, FOXM1. AURKA transactivates the FOXM1 promoter and stabilizes FOXM1 in late M phase and early G1 phase of the cell cycle, thus promoting proliferation in TNBC. AURKA overexpression in human cancers has been correlated with increased aneuploidy and centrosome amplification. Genomic instability can be induced in several cancers, including breast cancer, by increasing the level of AURKA. Results of whole genome and RNA sequencing of metastatic TNBC demonstrated co-over-expression of FOXM1 and AURKA in TNBCs, in association with other markers of rapid proliferation. In addition, AURKA was reported to phosphorylate YAP and to promote YAP⁻ mediated transcription in TNBC. A study of archival formalin-fixed paraffin-embedded (FFPE) TNBC tissue has also shown overexpression of FOXM1 and AURKA. Overexpression of AURKA has been shown to be prognostic in both of ER+/HER2− and TNBC subtypes. Two retrospective studies showed that high expression of AURKA was associated with shorter recurrence-free and overall survival in early stage TNBC and in node-negative ER+/HER2− patients.

Alisertib (MILN8237) is an ATP-competitive and reversible inhibitor of Aurora A kinase with an in vitro inhibition constant (Ki) of 0.43 nM. Alisertib has been evaluated as a single agent in a phase 1/2 study in adults with solid tumors, and the drug proved to inhibit the interaction between N-myc and its stabilizing factor AURKA, inhibiting N-myc signaling and suppressing tumor growth. Treatment of tumor cell lines with alisertib induced phenotypes consistent with AURKA inhibition, including mitotic spindle defects, mitotic delay, and apoptosis. Synergistic or additive effects have been observed in breast cancer xenograft models when alisertib was added to either paclitaxel or docetaxel. In addition, alisertib inhibited Pgp-mediated efflux of paclitaxel in a cell culture model. The combination of paclitaxel with alisertib (P+A) has also been investigated in a phase I study in patients with locally advanced or metastatic ovarian or breast cancers, with preliminary evidence of activity in both tumor types including 6 partial responses and 3 stable disease in 11 patients with MBC. Adverse events (AE) observed with alisertib in combination with paclitaxel included stomatitis, neutropenia, leukopenia, anemia, fatigue, diarrhea, headache, nausea, and pleural effusion.

### SUMMARY

This disclosure is based on the surprising discovery that a combination of alisertib and paclitaxel is significantly more effective in treating certain patients with breast cancers (e.g., ER+ and HER2− breast cancer or TN breast cancer) than paclitaxel alone.

In one aspect, this disclosure features a method of treating cancer that includes administering to a patient in need thereof therapeutically effective amounts of a combination of alisertib and paclitaxel, in which the cancer is estrogen receptor-positive and HER2-negative breast cancer and the patient has been treated with a CDK 4/6 inhibitor before being treated with the combination of alisertib and paclitaxel.

In another aspect, this disclosure features a method of treating cancer that includes administering to a patient in need thereof therapeutically effective amounts of a combination of alisertib and paclitaxel, in which the cancer is triple-negative breast cancer.

Other features, objects, and advantages will be apparent from the description and the claims.

### DESCRIPTION OF DRAWINGS

FIG. 1 shows a study flow diagram of the clinical trial described in Example 1.

FIG. 2 shows estimates of progression-fee survival in the ER+/HER2− intent-to-treat population in the clinical trial described in Example 1 (P=Paclitaxel, P+A=Paclitaxel+Alisertib).

FIG. 3 shows estimates of progression-free survival in the TN intent-to-treat population in the clinical trial described in Example 1 (P=Paclitaxel, P+A=Paclitaxel+Alisertib).

Like reference symbols in the various drawings indicate like elements.

### DETAILED DESCRIPTION

This disclosure generally relates to methods of treating breast cancer by using a combination of alisertib and paclitaxel.

### Combination Therapy

Alisertib (i.e., 4-{[9-chloro-7-(2-fluoro-6-methoxyphenyl)-5H-pyrimido[5,4-d][2]benzazepin-2-yl]amino}-2-methoxybenzoic acid) is a known selective aurora A kinase inhibitor, and has the following chemical structure:

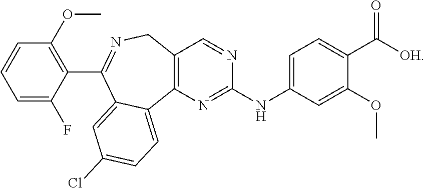 [SMILES: COc1cc(Nc2ncc3c(n2)-c2ccc(Cl)cc2C(c2c(F)cccc2OC)=NC3)ccc1C(=O)O]

Paclitaxel (i.e., (2α,4α,5β,7β,10β,13α)-4,10-Bis(acetyloxy)-13-{[(2R,3S)-3-(benzoylamino)-2-hydroxy-3-phenylpropanoyl]oxy}-1,7-dihydroxy-9-oxo-5,20-epoxytax-11-en-2-yl benzoate) is a known microtubule inhibitor that can be used to treat a number of cancers (e.g., including ovarian cancer, esophageal cancer, and breast cancer). Paclitaxel has the following chemical structure:

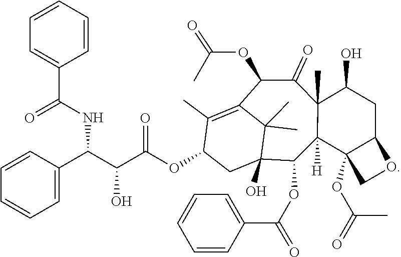 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Without wishing to be bound by theory, it is believed that a combination of alisertib and paclitaxel is significantly more effective in treating breast cancers (e.g., ER+ and HER2− breast cancer or TN breast cancer) than paclitaxel alone.

As used herein, the term “alisertib” include alisertib free acid (i.e., 4-{[9-chloro-7-(2-fluoro-6-methoxyphenyl)-5H-pyrimido[5,4-d][2]benzazepin-2-yl]amino}-2-methoxybenzoic acid), a pharmaceutically acceptable salt of alisertib free acid, or a hydrate (e.g., a monohydrate) of alisertib free acid or a pharmaceutically acceptable salt thereof. A pharmaceutically acceptable salt, for example, can be formed between an anion and a positively charged group (e.g., amino) on alisertib. Suitable anions include chloride, bromide, iodide, sulfate, nitrate, phosphate, citrate, methanesulfonate, trifluoroacetate, acetate, malate, tosylate, tartrate, fumurate, glutamate, glucuronate, lactate, glutarate, and maleate. Likewise, a salt can also be formed between a cation and a negatively charged group (e.g., carboxylate) on alisertib. Suitable cations include sodium ion, potassium ion, magnesium ion, calcium ion, and an ammonium cation such as tetramethylammonium ion. For example, the combination described herein can include alisertib sodium monohydrate.

In some embodiments, alisertib can be administered orally to a patient. In such embodiments, alisertib can be administered at a dosage of from 30 mg to 50 mg (e.g., 40 mg) twice daily on days 1-3, 8-10, and 15-17 of a 28-day cycle. In some embodiments, the 28-day cycle regimen can repeat until the patient no longer needs this treatment. In some embodiments, alisertib can be administered at a different administration frequency (e.g., once daily, three times daily, or four times daily) and/or at a different schedule (e.g., on days 1-7 in 21-day cycles or on days 1-21 in 35-day cycles).

In some embodiments, paclitaxel can be administered intravenously to a patient. In such embodiments, paclitaxel can be administered at a dosage of 60 mg/m² over a suitable period of time (e.g., 1 hour) on days 1, 8, and 15 of a 28-day cycle. In some embodiments, the 28-day cycle regimen can repeat until the patient no longer needs this treatment. In some embodiments, paclitaxel can be administered at a different dosage (e.g., from 80 to 175 mg/m²) at a different schedule (e.g., once every week, once every two weeks, or once every three weeks). For example, paclitaxel can be administered 80 to 90 mg/m² over an hour per week, 100 mg/m² over 3 hours every two weeks, or 175 mg/m² over 3 hours every 3 weeks.

In some embodiments, alisertib and paclitaxel can be separately included in a pharmaceutical composition to be administered to a patient. For example, alisertib can be included in a pharmaceutical composition (e.g., a tablet or capsule) suitable for oral administration. As another example, paclitaxel can be included in a pharmaceutical composition (e.g., a solution) suitable for intravenous administration.

In some embodiments, the pharmaceutical compositions described herein can include a therapeutically effective amount of an active ingredient (e.g., alisertib or paclitaxel) and at least one pharmaceutically acceptable carrier (e.g., adjuvant or diluent). The carrier in the pharmaceutical compositions must be “acceptable” in the sense that it is compatible with the active ingredient of the composition (and preferably, capable of stabilizing the active ingredient) and not deleterious to the patient to be treated. One or more solubilizing agents can be utilized as pharmaceutical carriers for delivery of the active ingredients described herein. Examples of other carriers include colloidal silicon oxide, magnesium stearate, cellulose, sodium lauryl sulfate, and D&C Yellow #10. The pharmaceutical composition described herein can optionally include at least one further additive selected from a disintegrating agent, binder, lubricant, flavoring agent, preservative, colorant and any mixture thereof. Examples of such and other additives can be found in “Handbook of Pharmaceutical Excipients”; Ed. A. H. Kibbe, 3rd Ed., American Pharmaceutical Association, USA and Pharmaceutical Press UK, 2000.

A pharmaceutical composition for oral administration can be any orally acceptable dosage form including capsules, tablets, emulsions and aqueous suspensions, dispersions, and solutions. In the case of tablets, commonly used carriers include lactose and corn starch. Lubricating agents, such as magnesium stearate, are also typically added. For oral administration in a capsule form, useful diluents include lactose and dried corn starch. When aqueous suspensions or emulsions are administered orally, the active ingredient can be suspended or dissolved in an oily phase combined with emulsifying or suspending agents. If desired, certain sweetening, flavoring, or coloring agents can be added.

The pharmaceutical compositions described herein can be adapted for parenteral, oral, topical, nasal, rectal, buccal, or sublingual administration or for administration via the respiratory tract, e.g., in the form of an aerosol or an air-suspended fine powder. The term “parenteral” as used herein refers to subcutaneous, intracutaneous, intravenous, intramuscular, intraarticular, intraarterial, intrasynovial, intrasternal, intrathecal, intralesional, intraperitoneal, intraocular, intra-aural, or intracranial injection, as well as any suitable infusion technique. In some embodiments, the composition can be in the form of tablets, capsules, powders, microparticles, granules, syrups, suspensions, solutions, nasal spray, transdermal patches, injectable solutions, or suppositories.

A sterile injectable composition can be a solution or suspension in a non-toxic parenterally acceptable diluent or solvent, such as a solution in 1,3-butanediol. Among the acceptable vehicles and solvents that can be employed are mannitol, water, Ringer's solution, and isotonic sodium chloride solution. In addition, fixed oils are conventionally employed as a solvent or suspending medium (e.g., synthetic mono- or diglycerides). Fatty acid, such as oleic acid and its glyceride derivatives are useful in the preparation of injectables, as are natural pharmaceutically acceptable oils, such as olive oil or castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens or Spans or other similar emulsifying agents or bioavailability enhancers which are commonly used in the manufacture of pharmaceutically acceptable solid, liquid, or other dosage forms can also be used for the purpose of formulation.

### Methods of Treatment

In some embodiments, this disclosure features a method of using a combination of alisertib and paclitaxel for treating breast cancer in a patient (e.g., a human patient) in need of such treatment. The method can include administering to a patient in need thereof a combination of alisertib and paclitaxel in amounts therapeutically effective to treat the breast cancer. As used herein, “a therapeutically effective amount” refers to the amount of each active ingredient (i.e., alisertib and paclitaxel) that is required to confer a therapeutic effect on the treated patient.

As used herein, the terms “treatment,” “treat,” and “treating” refer to reversing, alleviating, delaying the onset of, or inhibiting the progress of, a cancer or one or more symptoms thereof, as described herein. In some embodiments, treatment can be administered after one or more symptoms have developed. In other embodiments, treatment can be administered in the absence of symptoms. For example, treatment can be administered to a susceptible individual prior to the onset of symptoms (e.g., in light of a history of symptoms and/or in light of genetic or other susceptibility factors). Treatment can also be continued after symptoms have resolved, for example to prevent or delay their recurrence.

In some embodiments, the breast cancer described herein can be estrogen receptor-positive (ER+) and HER2-negative breast cancer. In some embodiments, ER+ and HER2− breast cancer can be metastatic. In some embodiments, patients having ER+ and HER2− breast cancer can have at least one (e.g., two or three) pretreatment with a drug (e.g., a chemotherapy drug) before being treated with the combination of alisertib and paclitaxel. The pretreatment can include an endocrine or hormone therapy, or treatment with a CDK 4/6 inhibitor (e.g., palbociclib, ribociclib, or abemaciclib) and/or an mTOR inhibitor (e.g., everolimus). The endocrine or hormone therapy can include treatment with an aromatase inhibitor (e.g., anastrozole, exemestane, and letrozole), a selective estrogen receptor modulator (e.g., tamoxifen or toremifene), or an estrogen receptor degrader (e.g., fulvestrant). In some embodiments, patients having ER+ and HER2− breast cancer can be resistant to the above-mentioned pretreatments. Without wishing to be bound by theory, it is believed that the combination of alisertib and paclitaxel can have significantly improved efficacy than paclitaxel alone in treating patients having ER+ and HER2− breast cancer (e.g., patients having no pretreatment or at least one of the above-mentioned pretreatments).

In some embodiments, the breast cancer described herein can be triple negative breast cancer (TNBC). In some embodiments, TNBC can be metastatic. In some embodiments, patients having TNBC can have at least one (e.g., two or three) pretreatment with a drug (e.g., a chemotherapy drug), such as a platinum-based regimen, before being treated with the combination of alisertib and paclitaxel. In some embodiments, patients having TNBC can be resistant to the above-mentioned pretreatments. In some embodiments, the combination of alisertib and paclitaxel can be the first or initial treatment of patients having TNBC (i.e., patients having no pretreatment). Without wishing to be bound by theory, it is believed that the combination of alisertib and paclitaxel can have significantly improved efficacy than paclitaxel alone in treating patients having TNBC (e.g., patients having no pretreatment or at least one of the above-mentioned pretreatments).

In some embodiments, the treatment method described herein can further include identifying a patient having a breast cancer (e.g., an ER+ and HER2− breast cancer or TNBC). For example, to identify a patient having an ER+ and HER2− breast cancer, the treatment method described herein can include: (1) obtaining a biological sample containing cells (e.g., cells from breast or a different organ) from the patient (e.g., through biopsy); (2) assaying the sample to determine whether the sample has estrogen receptor expression or over expression of HER2 protein; and (3) based on a result of the assaying, determining that the patient has an estrogen receptor-positive and HER2− negative breast cancer. As used herein, “over expression of HER2 protein” refers to the amount of HER2 protein in a cancer tissue higher than the amount of HER2 protein in a normal tissue in the same organ (e.g., breast). The assays for determining whether a sample containing cells having estrogen receptors, progesterone receptors, or over expression of HER2 protein are well known in the art and can include an immunohistochemistry (IHC) test or a fluorescence in situ hybridization (FISH) test. In some embodiments, if the cells in the biological sample are obtained from an organ different from breast and are ER+ and HER2−, a sample containing breast cells can be obtained and assayed to confirm that the patient has an ER+ and HER2− breast cancer.

As another example, to identify a patient having TNBC, the treatment method described herein can include: (1) obtaining a biological sample containing cells (e.g., cells from breast or a different organ) from the patient (e.g., through biopsy); (2) assaying the sample to determine whether the sample has estrogen receptor expression, progesterone receptor expression, or over expression of HER2 protein; and (3) based on a result of the assaying, determining that the patient has a triple-negative breast cancer. The assays for determining whether a sample containing cells having estrogen receptors, progesterone receptors, or over expression of HER2 protein are well known in the art and can include an immunohistochemistry (IHC) test or a fluorescence in situ hybridization (FISH) test. In some embodiments, if the cells in the biological sample are obtained from an organ different from breast and are triple-negative, a sample containing breast cells can be obtained and assayed to confirm that the patient has an TN breast cancer.

The following examples are illustrative and not intended to be limiting.

### EXAMPLE

### Example 1: Phase II Clinical Trial of Alisertib and Paclitaxel Combination

This randomized, phase II study was conducted to assess the effectiveness and safety of the addition of alisertib to weekly paclitaxel therapy in patients with ER+/HER2− breast cancer or patients with TN breast cancer and was based on the need for effective strategies to prevent or delay the emergence of resistance to taxane therapy in the metastatic setting. The primary objective of the study was to demonstrate the superiority of the combination of paclitaxel and alisertib (P+A) compared with paclitaxel-alone (P-alone) in progression-free survival (PFS) in the two cohorts of metastatic breast cancer (MBC) patients. Other information about this phase II clinical trial is included in O'Shaughnessy et al., JAMA Network Open. 2021, 4(4):e214103, the entire contents of which are incorporated herein by reference.

### Methods

This randomized phase II trial with a concurrent control arm was designed to demonstrate superior PFS with paclitaxel plus alisertib compared to paclitaxel alone in patients with ER-positive, HER2-negative (ER+/HER2−), or with TN metastatic or locally recurrent breast cancer. The study also assessed the safety of paclitaxel plus alisertib, compared the objective response rates (ORR), complete response (CR), partial response (PR), and clinical benefit rates (CBR; defined as CR+PR+stable disease (SD)≥6 months) associated with paclitaxel plus alisertib vs paclitaxel alone, and assessed the overall survival (OS) in each of the treatment arms in the two separate patient cohorts (ER+/HER2− and TN). In addition, the patients' primary or metastatic formalin-fixed, paraffin-embedded breast cancer tissue was archived to identify potential biomarkers for alisertib benefit.

Eligible patients were postmenopausal women aged 18 years or older with metastatic or unresectable locally recurrent breast cancer that was histologically confirmed as ER+/HER2− invasive breast cancer (any progesterone receptor status), with Ki67 greater than 15% in primary or metastatic tissue, or grade 3 TNBC, to enrich for more highly proliferative cancers that may be more dependent on AURKA. HER2− negative was defined as immunohistochemistry (IHC) status of 0, 1+, or 2+(if IHC was 2+, a negative FISH test was required with a ratio <2.0 and an average HER2 copy number <4.0 signals/cell). ER-negative and progesterone receptor (PR)-negative status was defined as ER and PR less than 1% nuclei positive by IHC. Patients may have been treated with 0 or 1 chemotherapy regimen for advanced disease and with neo/adjuvant taxane therapy at least 12 months before the development of metastatic disease. An Eastern Cooperative Group (ECOG) performance status of 0 or 1 and adequate hematologic, cardiac, renal, and liver function were required. Eligible patients were required to have measurable disease by Response Evaluation Criteria in Solid Tumors (RECIST v 1.1) criteria, or lytic or mixed blastic/lytic bone-only disease. Patients with untreated or progressing brain metastases, or those who required corticosteroids, were not eligible.

Patients were randomly assigned to receive either paclitaxel 60 mg/m² intravenously (IV) on days 1, 8 and 15 of a 28-day cycle plus alisertib 40 mg twice daily (BID) on days 1-3, 8-10, and 15-17 of a 28-day cycle or to receive single-agent paclitaxel 90 mg/m² IV on days 1, 8 and 15 of a 28-day cycle. Administration of 5 μg/kg filgrastim was allowed for treatment-limiting neutropenia, and loperamide was used to treat diarrhea but was not allowed prophylactically in cycle 1.

Computed tomography (CT) of chest, abdomen, and pelvis was performed at screening. Chest and abdominal CT imaging were then performed every 8 weeks after initiation of treatment for the first 32 weeks of treatment and every 12 weeks thereafter for the duration of the treatment period, and at End of Treatment (EoT). Pelvic CT imaging was obtained at subsequent tumor assessments only if pelvic metastases were identified at screening. Bone scans were obtained on all patients at screening and then every 12 weeks in patients with bone metastases on their baseline bone scan.

Safety was assessed throughout the study. Adverse events were graded and reported according to the National Cancer Institute Common Terminology Criteria for Adverse Events (CTCAE version 4.03).

### Statistical Analysis

This open-label, randomized phase II clinical trial was conducted to study the effectiveness of alisertib plus paclitaxel in improving PFS compared with paclitaxel alone in two separate cohorts of patients, i.e., (2) ER+/HER2− breast cancer patients or (2) TN metastatic or locally recurrent breast cancer patients. Patients were stratified as ER-positive, HER2-negative cohort and TN cohort based on results of ER, progesterone receptor, and HER2 measurements on their metastatic tissue, or, if not available, on their primary breast cancer tissue. For each cohort, a blocked randomization table with the block size of 4 was created by the US Oncology Research biostatistician, and patients were randomly assigned to receive either paclitaxel alone or paclitaxel plus alisertib.

It was estimated that median PFS in patients with TN and ER-positive, HER2− negative MBC treated with paclitaxel alone would be 6 months for first-line treatment and 3.5 months for second line treatment. Using a 2-stage, sequential design with futility boundaries for each group, 112 patients with TN MBC and 140 patients with ER-positive, HER2-negative breast cancer were required to demonstrate a hazard ratio of 0.6 with 80% power and 1-sided a of 5% for PFS with the addition of alisertib to paclitaxel in each of the 2 cohorts. The TN and ER-positive, HER2-negative cohorts were analyzed independently, and no change to the statistical plan was made based on the underaccrual to the TN group.

Patients' baseline demographic and disease characteristics were summarized for the intent-to-treat population, which included all patients registered on study and were described by means with SDs and medians with ranges for continuous variables and by numbers with percentages for categorical variables. PFS was defined as the time from date of registration in the study to the date of first progression of disease or the date of death. Patients whose disease had not progressed were censored at the last contact date. OS was measured from the date of registration to the date of death. PFS and OS were estimated using Kaplan-Meier methods with 95% CIs in the intent-to-treat population and compared by log-rank tests.

The protocol-defined evaluable population included all eligible patients who received at least 1 dose of study drug. CBR and ORR were examined using univariate and multivariate approaches with Fisher exact tests and the logistic regression models. The 95% CIs were estimated for CBR and ORR assuming binomial distribution. A χ² test was used to estimate the differences between CBRs and ORRs in the two arms.

Incidence and grades of AEs for all patients were analyzed using the safety population which included all patients (eligible and ineligible) who received at least 1 dose of study drug. Toxic effects were reported and tabulated by numbers and frequencies for all related AEs. SAS statistical software version 9.4 (SAS Institute) was used for all data analysis. Data were analyzed from March through May 2019.

### Results

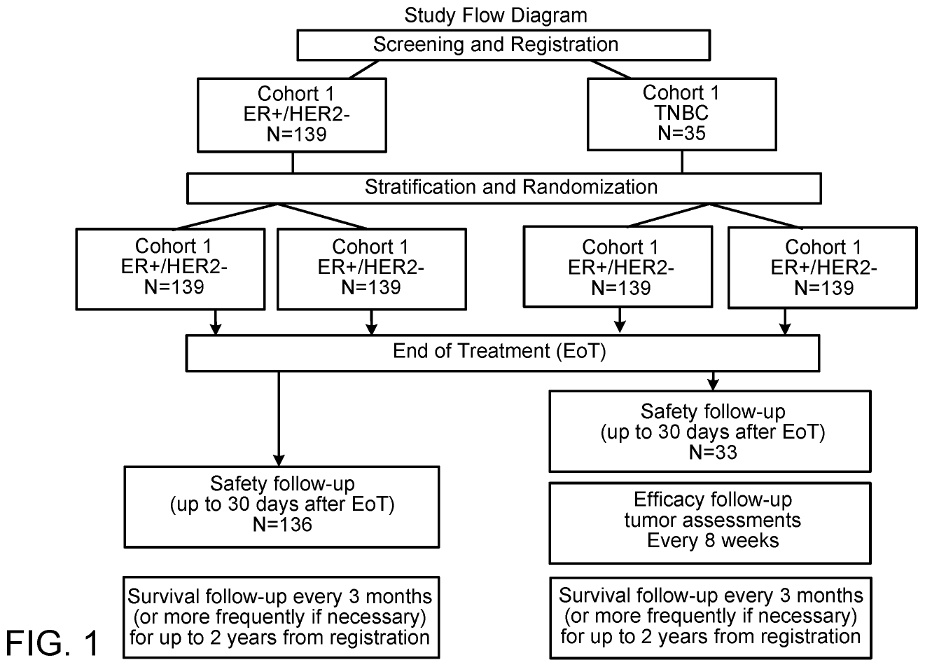

Between February 2015 and February 2018, a total of 174 women were enrolled in the trial and were randomly assigned to paclitaxel alone (86 patients), or paclitaxel plus alisertib (88 patients) cohorts. Only women were included in the trial, as MBC is very rare among men, and the safety and efficacy of combination therapy strategies is poorly understood in this population. The trial enrolled 139 patients with ER-positive, HER2-negative breast cancer, with 70 randomized to paclitaxel and 69 randomized to paclitaxel plus alisertib. The TN cohort closed with only 35 patients enrolled owing to slow accrual. The study flow diagram is summarized in FIG. 1. In the ER-positive, HER2-negative group, the patient characteristics were well balanced between the two treatment arms and are summarized in Table 1 below. The median (range) age was 62 (27-84) years, with 82 patients (59.0%) older than 65 years. A total of 42 patients (30.2%) had received prior chemotherapy for metastatic disease, 57 patients (41.0%) had received prior neo or adjuvant taxane therapy, and 75 patients (54.0%) had a disease-free interval of 5 or more years. Overall, 126 patients (90.6%) had been previously treated with an aromatase inhibitor, 40 patients (28.8%) had been treated with fulvestrant, 44 patients (31.7%) had been treated with tamoxifen, 19 patients (13.7%) had been treated with everolimus, and 28 patients (20.1%) had been treated with palbociclib. The main sites of metastatic disease were bone and liver. There were 3 patients who did not receive any study drug owing to prolonged thrombocytopenia, withdrawal of consent, or new brain metastases.

TABLE 1
Demographics and Baseline Characteristics of ER+/HER2−
Cohort (Intent-to-Treat Population)

| | | P + A | | P | |
|---|---|---|---|---|---|
| Parameter | | n = 69 (%) | | n = 70 (%) | |
| Age, years (range) | Median | 62.5 | (33.1, 83.4) | 63.2 | (26.9, 84.2) |
| Race/ethnicity | ASIAN | 1 | (1.4) | 1 | (1.4) |
| | BLACK | 7 | (10.1) | 6 | (8.6) |
| | CAUCASIAN | 54 | (78.3) | 55 | (78.6) |
| | HISPANIC | 6 | (8.7) | 6 | (8.6) |
| | OTHER | 1 | (1.4) | 2 | (2.9) |
| ECOG Performance Status | 0 | 36 | (52.2) | 31 | (44.3) |
| | 1 | 32 | (46.4) | 39 | (55.7) |
| | Missing | 1 | (1.4) | 0 | |
| Current Histological | Ductal | 45 | (65.2) | 47 | (67.1) |
| Findings | Lobular | 9 | (13.0) | 10 | (14.3) |
| | Mixed ductal and | 2 | (2.9) | 2 | (2.9) |
| | lobular | | | | |
| | Other | 13 | (18.8) | 11 | (15.7) |
| Positive Nodes at | 0 | 33 | (47.8) | 36 | (51.4) |
| Diagnosis, No. | 1 to 3 | 20 | (29.0) | 20 | (28.6) |
| | ≥4 | 13 | (18.8) | 9 | (12.9) |
| | Missing | 3 | (4.3) | 5 | (7.1) |
| Prior Chemotherapy for | 0 | 48 | (69.6) | 48 | (68.6) |
| Metastatic Disease, No. | | | | | |
| | 1 | 21 | (30.4) | 22 | (31.4) |
| Prior Neo/Adjuvant Taxane | No | 40 | (58.0) | 42 | (60.0) |
| | Yes | 29 | (42.0) | 28 | (40.0) |
| Prior Paclitaxel or | Paclitaxel | 17 | (24.6) | 12 | (17.1) |
| Docetaxel | Docetaxel | 16 | (23.2) | 20 | (28.6) |
| Disease-free Interval | <5-year | 24 | (46.2) | 22 | (44.9) |
| (in non-de | | | | | |
| novo MBC patients) | ≥5-year | 28 | (53.8) | 27 | (55.1) |
| Site of Metastasis | Bone | 45 | (65.2) | 55 | (78.6) |
| | Lymph node/Soft | 25 | (36.2) | 23 | (32.9) |
| | tissue | | | | |
| | Visceral-Liver | 39 | (56.5) | 36 | (51.4) |
| | Visceral-Lung | 15 | (21.7) | 19 | (27.1) |
| | Visceral-Others | 8 | (11.6) | 13 | (18.6) |
| | Visceral- | 0 | | 1 | (1.4) |
| | Peritoneum | | | | |
| | Brain | 1 | (1.4) | 1 | (1.4) |
| | Bone | 9 | (13.0) | 9 | (12.9) |

The patient characteristics for the patients with TN MBC are summarized in Table 2 below. Median (range) age was 65 (27-84) years, 11 patients (31.4%) had received prior chemotherapy for metastatic disease, 23 patients (65.7%) had received neo or adjuvant taxane therapy, and 17 patients (58.60%) had a disease-free interval less than 5 years. Two patients never received study treatment owing to a local recurrence or hospitalization.

TABLE 2
Demographics and Baseline Characteristics
for TN Cohort (Intent-to-Treat Population)

| | | P + A | | P | |
|---|---|---|---|---|---|
| Parameter | | n = 19 (%) | | n = 16 (%) | |
| Age | Median (Range) | 63.2 | (38.8, 75.0) | 65.2 | (53.1, 81.3) |
| Race | ASIAN | 0 | | 1 | (6.3) |
| | BLACK | 4 | (21.1) | 6 | (37.5) |
| | CAUCASIAN | 13 | (68.4) | 9 | (56.3) |
| | HISPANIC | 2 | (10.5) | 0 | |
| ECOG Performance | 0 | 8 | (42.1) | 9 | (56.3) |
| Status | 1 | 11 | (57.9) | 6 | (37.5) |
| | 2 | 0 | | 1 | (6.3) |
| Current Histology | Ductal | 16 | (84.2) | 11 | (68.8) |
| | Lobular | 0 | | 1 | (6.3) |
| | Other | 2 | (10.5) | 4 | (25.0) |
| | Missing | 1 | (5.3) | 0 | |
| Positive Nodes | 0 | 9 | (47.4) | 7 | (43.8) |
| at diagnosis | 1-3 | 4 | (21.1) | 3 | (18.8) |
| | ≥4 | 5 | (26.3) | 5 | (31.3) |
| | Missing | 1 | (5.3) | 1 | (6.3) |
| Prior Chemotherapy | 0 | 13 | (68.4) | 11 | (68.8) |
| for Metastatic Disease | | | | | |
| | 1 | 6 | (31.6) | 5 | (31.3) |
| Prior Neo/Adjuvant | No | 7 | (36.8) | 5 | (31.3) |
| Taxane | Yes | 12 | (63.2) | 11 | (68.8) |
| Prior Paclitaxel or | Paclitaxel | 5 | (26.3) | 5 | (31.3) |
| Docetaxel | Docetaxel | 7 | (36.8) | 7 | (43.8) |
| Disease-free Interval | <5-year | 9 | (56.3) | 8 | (61.5) |
| (among non-de novo | | | | | |
| metastatic patients) | ≥5-year | 7 | (43.8) | 5 | (38.5) |
| Site of Metastasis | Bone | 8 | (42.1) | 8 | (50.0) |
| | Lymph node/Soft | 11 | (57.9) | 11 | (68.8) |
| | tissue | | | | |
| | Liver | 3 | (15.8) | 6 | (37.5) |
| | Lung | 8 | (42.1) | 10 | (62.5) |
| | Others | 3 | (15.8) | 7 | (43.8) |

| | | | | |
|---|---|---|---|---|
| Bone only Metastasis | Bone | 2 | (10.5) | 0 |

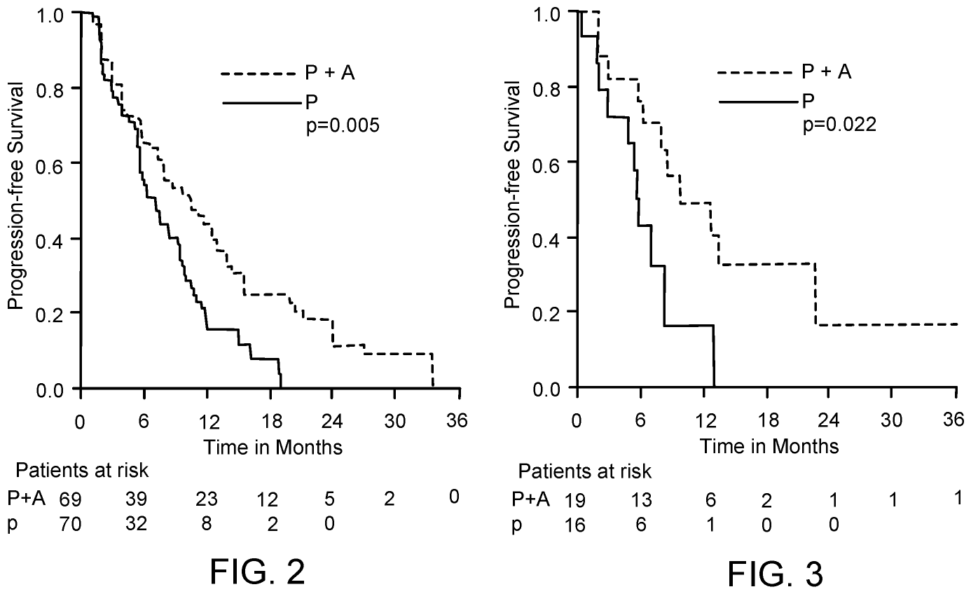

In the patients with ER-positive, HER2-negative MBC, the median interquartile range (IQR) PFS was 10.2 (3.8-15.7) months with paclitaxel plus alisertib vs 7.1 (3.8-10.6) months with paclitaxel alone (HR, 0.56; 95% CI, 0.37-0.84; P=0.005) (FIG. 2). The estimated PFS at 12 months was 44.0% (95% CI, 30.9%-56.3%) with paclitaxel plus alisertib and 15.4% (95% CI, 7.3%-26.1%) with paclitaxel alone. With a median (IQR) follow-up time of 22 (10.6-25.1) months, the median (IQR) OS was 26.3 (12.4-37.2) months with paclitaxel plus alisertib vs 25.1 (11.0-31.4) months with paclitaxel alone (HR, 0.89; 95% CI, 0.58-1.38; P=0.61). In 30 patients who had been previously treated with palbociclib for MBC, the median (IQR) PFS with paclitaxel alone was 5.6 (3.0-10.6) months (16 patients) and with paclitaxel plus alisertib was 13.9 (5.6-15.6) months (14 patients) (HR, 0.58; 95% CI, 0.26-1.32; P=0.19). The CBR observed with paclitaxel plus alisertib in patients who had been pretreated with palbociclib was 61.5% (95% CI, 31.6%-86.1%) vs 37.5% (95% CI, 15.2%-64.6%) in patients who had received paclitaxel alone.

In the ER-positive, HER2-negative cohort, the ORR was 31.0% (95% CI, 19.5%-44.5%) in the paclitaxel plus alisertib group vs 33.9% (95% CI, 22.3%-47.0%) in the paclitaxel alone group (Table 3). The CBR was 67.2% (95% CI, 53.7%-79.0%) in the paclitaxel plus alisertib arm and 56.5% (95% CI, 43.3%-69.0%) in the paclitaxel alone group (Table 3).

TABLE 3
Overall Response Rates by Study Treatment
in the ER+/HER2− Cohort

| | P + A | | | P | |
|---|---|---|---|---|---|
| | (n = 58) | | | (n = 62) | |
| Best Overall Response | n (%) | | 95% CI | n (%) | 95% CI |
| CR | 1 | (1.7) | 0-9.2 | 0 | NA |
| PR | 17 | (29.3) | 18.1-42.7 | 21 (33.9) | 22.3-47.0 |
| ORR (CR + PR)* | 18 | (31.0) | 19.5-44.5 | 21 (33.9) | 22.3-47.0 |
| SD | 35 | (60.3) | 46.6-73.0 | 33 (53.2) | 40.1-66.0 |
| SD ≥ 6 months | 21 | (36.2) | 24.0-49.9 | 14 (22.6) | 12.9-35.0 |
| CBR (CR + PR + | 39 | (67.2) | 53.7-79.0 | 35 (56.5) | 43.3-69.0 |
| SD ≥ 6 months)* | | | | | |
| PD | 5 | (8.6) | 2.9-19.0 | 8 (12.9) | 5.7-23.9 |

Abbreviation:
CR, complete response;
NA, not applicable;
PR, partial response;
ORR, objective response rate;
SD, stable disease;
CBR, clinical benefit rate;
PD, progression of disease
*p > 0.05 between intervention and control

Among 35 patients with TN MBC, the median (IQR) PFS was 9.6 (6.1-22.6) months with paclitaxel plus alisertib vs 5.7 (2.9-8.2) months with paclitaxel alone (HR, 0.35; 95% CI, 0.14-0.89; P=0.02) (FIG. 3). With a median (IQR) follow-up of 13.7 (7.5-23.7) months, the median (IQR) OS was 16 (9.6-34.0) months with paclitaxel plus alisertib vs 12.7 (6.8-23.5) months with paclitaxel alone (HR, 0.51; 95% CI, 0.23-1.13; P=0.09). The ORRs and CBRs in the evaluable patients with TNBC are summarized in Table 4 below.

TABLE 4
Overall Response Rate by Study Treatment in TN Cohort

| | P + A | | | P | |
|---|---|---|---|---|---|
| | (n = 16) | | | (n = 12) | |
| Best Overall Response | n (%) | | 95% CI | n (%) | 95% CI |
| CR | 1 | (6.3) | 0.2-30.2 | 0 | NA |
| PR | 8 | (50.0) | 24.7-75.3 | 5 (41.7) | 15.2-72.3 |
| ORR (CR + PR) | 9 | (56.3) | 29.9-80.2 | 5 (41.7) | 15.2-72.3 |
| SD | 5 | (31.3) | 11.0-58.7 | 5 (41.7) | 15.2-72.3 |
| SD ≥ 6 months | 4 | (25.0) | 7.3-52.4 | 2 (16.7) | 2.1-48.4 |
| CBR (CR + PR + | 13 | (81.3) | 54.4-96.0 | 7 (58.3) | 27.7-84.8 |
| SD ≥ 6 months)* | | | | | |
| PD | 2 | (12.5) | 1.6-38.3 | 2 (16.7) | 2.1-48.4 |

*p-value = 0.23 between intervention and control

In the ER-positive, HER2-negative group treated with paclitaxel alone, 22 patients (31.4%) had a dose delay and dose reduction, while 41 patients (62.1%) had an alisertib dose delay and reduction and 42 patients (63.6%) had a paclitaxel dose delay in the paclitaxel plus alisertib group (Table 5). Similar findings were observed in the patients with TN MBC.

TABLE 5
Summary of Drug Exposure in Estrogen Receptor-Positive,
ERBB2-Negative Cohort (Safety Population)

| | | Arm 1 | | | | Arm 2 | |
|---|---|---|---|---|---|---|---|
| Parameter | | Alisertib | | Paclitaxel | | Paclitaxel | |
| Cycle | n | 66 | | 66 | | 70 | |
| | Mean (SD) | 8.1 | (7.3) | 9.6 | (8.6) | 6.5 | (4.7) |
| | Median (Range) | 5.0 | (1.0-30.0) | 7.0 | (1.0-36.0) | 6.0 | (1.0-21.0) |
| Drug Delivered per Dose | n | | | 66 | | 70 | |
| | Mean (SD) | | | 59.5 | (6.5) | 83.4 | (8.0) |
| | Median (Range) | | | 60.6 | (22.9-74.6) | 87.2 | (61.7-93.1) |
| Dose Intensity (%) | n | | | 66 | | 70 | |
| | Mean (SD) | | | 82.5 | (20.3) | 84.7 | (14.8) |
| | Median (Range) | | | 84.7 | (22.6-124) | 89.1 | (32.9-102) |
| Patients with dose delay | | 12 | (18.2) | 42 | (63.6) | 10 | (14.3) |
| alone [n (%)] | | | | | | | |
| Patients with dose | | 5 | (7.6) | 1 | (1.5) | 2 | (2.9) |
| reduction alone | | | | | | | |
| [n (%)] | | | | | | | |
| Patients with both dose | | 41 | (62.1) | 11 | (16.7) | 22 | (31.4) |
| delay and reduction | | | | | | | |
| [n (%)] | | | | | | | |
| Patients with dose | | 1 | (1.5) | 2 | (3.0) | 0 | |
| increased [n (%)] | | | | | | | |
| Patients with dose | | 20 | (30.3) | 11 | (16.7) | 8 | (11.4) |
| discontinuation [n (%)] | | | | | | | |
| Patient Compliance with | | 64.5 | (8.7) | | | | |
| Alisertib* [Mean % | | | | | | | |
| cycles (SD)] | | | | | | | |

*Compliance is based on 8 pills (80 mg) and 9 days (a total of 72 pills) per cycle.

Most of the adverse events (AEs) in all patients combined were grade 1 or 2. The main grade 3 or 4 adverse events with paclitaxel plus alisertib vs paclitaxel alone were neutropenia (50 patients [59.5%] vs 14 patients [16.4%]), anemia (8 patients [9.5%] vs 1 patient [1.2%]), diarrhea (9 patients [10.7%] vs 0 patients), stomatitis or oral mucositis (13 patients [15.5%] vs 0 patients) and neuropathy (1 patient [1.5%] vs 8 patients [11.40%]) (Table 6). Most of the serious AEs occurred in the paclitaxel plus alisertib group and included diarrhea (3 patients [4.50%]), febrile neutropenia (3 patients [4.5%]), sepsis (3 patients [4.50%]) or any of nausea, chest pain, peripheral neuropathy, pulmonary embolism, or respiratory infection (2 patients [3.00%] combined). One patient died from sepsis during paclitaxel plus alisertib treatment. A total of 29 patients (43.90%) with paclitaxel plus alisertib and 11 patients (15.70%) with paclitaxel alone received standard-dose (5 μg/kg) filgrastim.

TABLE 6
Treatment-related Toxic Effects in Both ER+/HER2− and TN Cohorts

| | P + A (n = 66) | | | | | P (n = 70) | | | | |
|---|---|---|---|---|---|---|---|---|---|---|
| Reported | Grade1 | Grade2 | Grade3 | Grade4 | Total | Grade1 | Grade2 | Grade3 | Grade4 | Total |
| Term | n (%) | n (%) | n (%) | n (%) | n (%) | n (%) | n (%) | n (%) | n (%) | n (%) |
| Neutropenia | 3 (3.6) | 4 (4.8) | 27 (32.1) | 23 (27.4) | 57 (67.9) | 1 (1.2) | 3 (3.5) | 11 (12.9) | 3 (3.5) | 18 (21.2) |
| Anemia | 8 (9.5) | 6 (7.1) | 8 (9.5) | 0 | 22 (26.2) | 5 (5.9) | 5 (5.9) | 1 (1.2) | 0 | 11 (12.9) |
| Leukopenia | 0 | 3 (3.6) | 5 (6.0) | 2 (2.4) | 10 (11.9) | 2 (2.4) | 1 (1.2) | 2 (2.4) | 0 | 5 (5.9) |
| Thrombo- | 3 (3.6) | 0 | 0 | 0 | 3 (3.6) | 0 | 0 | 0 | 0 | 0 |
| cytopenia | | | | | | | | | | |
| Febrile | 0 | 0 | 0 | 1 (1.2) | 1 (1.2) | 0 | 0 | 0 | 0 | 0 |
| neutropenia | | | | | | | | | | |
| Diarrhea | 17 (20.2) | 22 (26.2) | 9 (10.7) | 0 | 48 (57.1) | 9 (10.6) | 2 (2.4) | 0 | 0 | 11 (12.9) |
| Nausea | 24 (28.6) | 11 (13.1) | 0 | 0 | 35 (41.7) | 19 (22.4) | 4 (4.7) | 1 (1.2) | 0 | 24 (28.2) |
| Mucositis | 7 (8.3) | 7 (8.3) | 9 (10.7) | 0 | 23 (27.4) | 4 (4.7) | 0 | 0 | 0 | 4 (4.7) |
| oral | | | | | | | | | | |
| Stomatitis | 6 (7.1) | 4 (4.8) | 4 (4.8) | 0 | 14 (16.7) | 7 (8.2) | 0 | 0 | 0 | 7 (8.2) |
| Fatigue | 21 (25.0) | 17 (20.2) | 4 (4.8) | 0 | 42 (50.0) | 26 (30.6) | 6 (7.1) | 2 (2.4) | 0 | 34 (40.0) |
| Neuropathy | 7 (10.6) | 4 (6.1) | 1 (1.5) | 0 | 12 (18) | 9 (12.9) | 8 (11.4) | 8 (11.4) | 0 | 25 (35.7) |
| Dizziness | 7 (8.3) | 2 (2.4) | 0 | 0 | 9 (10.7) | 2 (2.4) | 0 | 0 | 0 | 2 (2.4) |
| Headache | 9 (10.7) | 2 (2.4) | 0 | 0 | 11 (13.1) | 4 (4.7) | 1 (1.2) | 0 | 0 | 5 (5.9) |

In this randomized clinical trial, patients received a lower-than-standard dose of weekly paclitaxel in combination with 3 days per week of alisertib to maximize concomitant exposure of alisertib and paclitaxel while providing treatment-free periods for recovery from neutropenia. The results show that, in 139 patients with ER-positive, HER2-negative MBC, the combination of alisertib and paclitaxel demonstrated a significant improvement in median PFS from 7.1 months with paclitaxel alone to 10.2 months when alisertib was added to paclitaxel. The PFS curves in the ER-positive, HER2-negative population separated at 6 months in the above study, suggesting that alisertib may delay the development of acquired resistance to paclitaxel. In addition, the combination of alisertib and paclitaxel significantly improved the median PFS (i.e., 13.9 months) compared to the median PFS (i.e., 5.6 months) achieved by paclitaxel alone in patient pretreated with a CNK 4/6 inhibitor (i.e., palbociclib).

Further, the results from the above study show that the combination of alisertib and paclitaxel demonstrated a significantly improved median PFS (i.e., 9.6 months) in treating patients with TN breast cancer compared to the median PFS (i.e., 5.7 months) achieved by paclitaxel alone.

Other embodiments are within the scope of the following claims.

What is claimed is:

1. A method of treating cancer, comprising:
administering to a patient in need thereof therapeutically effective amounts of a combination of alisertib and paclitaxel;
wherein the cancer is estrogen receptor-positive and HER2-negative breast cancer and the patient has been treated with a CDK 4/6 inhibitor before being treated with the combination of alisertib and paclitaxel.

2. The method of claim 1, further comprising identifying the patient as having an estrogen receptor-positive and ERBB2-negative breast cancer.

3. The method of claim 2, wherein the identifying comprises:
obtaining a biological sample containing cells from the patient;
assaying the sample to determine whether the sample has estrogen receptor expression or over expression of HER2 protein; and
based on a result of the assaying, determining that the patient has an estrogen receptor-positive and HER2-negative breast cancer.

4. The method of claim 1, wherein paclitaxel is administered intravenously.

5. The method of claim 4, wherein paclitaxel is administered at a dosage of 60 mg/m² on days 1, 8, and 15 of a 28-day cycle.

6. The method of claim 1, wherein alisertib is administered orally.

7. The method of claim 6, wherein alisertib is administered at a dosage of 40 mg twice daily on days 1-3, 8-10, and 15-17 of a 28-day cycle.

8. The method of claim 1, wherein the CDK 4/6 inhibitor is palbociclib, ribociclib, or abemaciclib.

9. The method of claim 1, wherein the breast cancer is resistant to the treatment of a CDK 4/6 inhibitor.

10. The method of claim 1, wherein the cancer is metastatic breast cancer.

11. A method of treating cancer, comprising:
administering to a patient in need thereof therapeutically effective amounts of a combination of alisertib and paclitaxel;
wherein the cancer is triple-negative breast cancer.

12. The method of claim 11, further comprising identifying the patient as having a triple-negative breast cancer.

13. The method of claim 12, wherein the identifying comprises:
obtaining a biological sample containing cells from the patient;
assaying the sample to determine whether the sample has estrogen receptor expression, progesterone receptor expression, or over expression of HER2 protein; and
based on a result of the assaying, determining that the patient has a triple-negative breast cancer.

14. The method of claim 11, wherein paclitaxel is administered intravenously.

15. The method of claim 14, wherein paclitaxel is administered at a dosage of 60 mg/m² on days 1, 8, and 15 of a 28-day cycle.

16. The method of claim 15, wherein alisertib is administered orally.

17. The method of claim 16, wherein alisertib is administered at a dosage of 40 mg twice daily on days 1-3, 8-10, and 15-17 of a 28-day cycle.

18. The method of claim 11, wherein the combination of alisertib and paclitaxel is an initial treatment to the patient.

19. The method of claim 11, wherein the patient has been treated with a drug before being treated with the combination of alisertib and paclitaxel.

20. The method of claim 11, wherein the cancer is metastatic breast cancer.
